(19) United States
(12) Patent Application Publication
(10) Pub. No.: US 2021/0321859 A1
(43) Pub. Date: Oct. 21, 2021

(54) ENDOSCOPE DEVICE

(71) Applicant: OLYMPUS CORPORATION, Tokyo (JP)

(72) Inventors: Takeshi MORI, Tokyo (JP); Katsunori KUMAI, Tokyo (JP); Takehiro MIKI, Tokyo (JP); Masashi YAMADA, Tokyo (JP)

(73) Assignee: OLYMPUS CORPORATION, Tokyo (JP)

(21) Appl. No.: 17/360,241

(22) Filed: Jun. 28, 2021

Related U.S. Application Data
(63) Continuation of application No. PCT/JP2019/000007, filed on Jan. 4, 2019.

(51) Int. Cl. A61B 1/00; A61B 1/07

(52) U.S. Cl. CPC A61B 1/00096; A61B 1/07

(57) ABSTRACT

Provided is a scanning endoscope including: an insertion portion that has a distal-end section and a proximal-end section; a light-guide optical system that guides illumination light toward the distal-end section; a spherical lens that is disposed in the distal-end section and that radiates the illumination light guided by the light-guide optical system onto a subject; an optical waveguide that extends from the distal-end section to the proximal-end section, that receives observation light coming from the subject, and that guides the observation light; and a light detector that detects the observation light guided by the optical waveguide, wherein the optical waveguide is inclined, at the distal-end section, in such a direction as to approach an optical axis of the spherical lens toward a distal end.

### CROSS-REFERENCE TO RELATED APPLICATIONS

This is a continuation of International Application PCT/JP2019/000007 which is hereby incorporated by reference herein in its entirety.

### TECHNICAL FIELD

The present invention relates to an endoscope device.

### BACKGROUND ART

In the related art, there is a known scanning-type endoscope device that scans illumination light on a subject by vibrating a distal-end section of an optical fiber and that forms an image of the subject on the basis of observation light from respective positions of the subject (for example, see PTL 1).

### CITATION LIST

### Patent Literature

{PTL 1} Japanese Unexamined Patent Application, Publication No. 2011-4929

### SUMMARY OF INVENTION

According to one aspect, the present invention provides a scanning endoscope including: a long insertion portion that has a distal-end section and a proximal-end section; a light-guide optical system that guides illumination light coming from a light source toward the distal-end section; a spherical lens that is disposed in the distal-end section and that radiates the illumination light guided by the light-guide optical system onto a subject; an optical waveguide that extends from the distal-end section to the proximal-end section, that receives observation light coming from the subject, and that guides the observation light; and a light detector that detects the observation light guided by the optical waveguide, wherein the optical waveguide is inclined, at the distal-end section, in such a direction as to approach an optical axis of the spherical lens toward a distal end.

According to another aspect, the present invention provides a scanning endoscope including: a long insertion portion that has a distal-end section and a proximal-end section; an optical waveguide that extends from the distal-end section to the proximal-end section, that guides illumination light coming from a light source toward the distal-end section, and that radiates the illumination light onto a subject; a spherical lens that is disposed in the distal-end section and that receives observation light coming from the subject; a light-guide optical system that guides the observation light received by the spherical lens; and a light detector that detects the observation light guided by the light-guide optical system, wherein the optical waveguide is inclined, at the distal-end section, in such a direction as to approach an optical axis of the spherical lens toward a distal end.

### BRIEF DESCRIPTION OF DRAWINGS

FIG. 1 is a view showing the overall configuration of an endoscope device according to one embodiment of the present invention.

FIG. 2A is a longitudinal sectional view of an illumination optical system and an optical waveguide in the endoscope device shown in FIG. 1.

FIG. 2B is a front view of the illumination optical system and the optical waveguide, which are shown in FIG. 2A, viewed from the distal end in the direction of the optical axis of the illumination optical system.

FIG. 3A is a view for explaining light-receiving ranges of the optical waveguide that has a tapered section.

FIG. 3B is a view for explaining light-receiving ranges of an optical waveguide according to a comparative example that does not have a tapered section.

FIG. 4A is a view for explaining design values of the optical waveguide that has the tapered section.

FIG. 4B is a view for explaining design values of the optical waveguide according to the comparative example that does not have a tapered section.

FIG. 5A is a side view showing a modification of the optical waveguide.

FIG. 5B is a front view of the illumination optical system and an optical waveguide that are shown in FIG. 5A, viewed from the distal end in the direction of the optical axis of the illumination optical system.

FIG. 6A is a side view showing another modification of the optical waveguide.

FIG. 6B is a front view of the illumination optical system and an optical waveguide that are shown in FIG. 6A, viewed from the distal end in the direction of the optical axis of the illumination optical system.

FIG. 7A is a longitudinal sectional view showing a modification of the illumination optical system and still another modification of the optical waveguide.

FIG. 7B is a view for explaining a light-receiving range of the optical waveguide shown in FIG. 7A.

FIG. 8A is a longitudinal sectional view showing another modification of the illumination optical system.

FIG. 8B is a longitudinal sectional view showing a modification of an insertion portion.

FIG. 8C is a longitudinal sectional view showing another modification of the insertion portion.

FIG. 9 is a longitudinal sectional view showing still another modification of the insertion portion.

FIG. 10A is a longitudinal sectional view showing still another modification of the illumination optical system.

FIG. 10B is a longitudinal sectional view showing still another modification of the illumination optical system.

### DESCRIPTION OF EMBODIMENT

An endoscope device 1 according to one embodiment of the present invention will be described below with reference to the drawings.

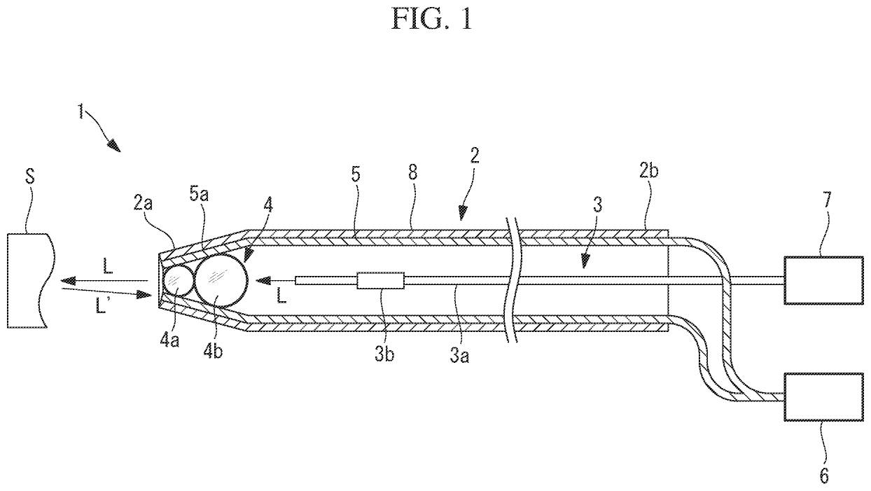

As shown in FIG. 1, the endoscope device 1 of this embodiment is a scanning-type endoscope device that scans illumination light L on a subject S. The endoscope device 1 includes: a long insertion portion 2 that has a distal-end section 2a and a proximal-end section 2b; a light-guide optical system 3 that guides the illumination light L coming from a light source 7, toward the distal-end section 2a; an illumination optical system 4 that is disposed in the distal-end section 2a and that radiates the illumination light L guided by the light-guide optical system 3 onto the subject S; an optical waveguide 5 that extends from the distal-end section 2a toward the proximal-end section 2b, that receives observation light L′ coming the subject S, and that guides the observation light L′; and a light detecting part 6 that detects the observation light L′ guided by the optical waveguide 5.

The insertion portion 2 includes a cylindrical rigid outer cover 8. The outer cover 8 is, for example, a pipe made of metal, such as stainless steel. The outer cover 8 is a member disposed at the radially outermost position of the insertion portion 2, and an outer circumferential surface of the outer cover 8 forms the outermost circumferential surface of the insertion portion 2. The distal-end section 2a is tapered so as to be gradually reduced in diameter toward the distal end.

The light-guide optical system 3 has an optical fiber 3a and a scanner 3b.

The optical fiber 3a is disposed inside the insertion portion 2 and extends along the longitudinal direction of the insertion portion 2. A proximal end of the optical fiber 3a is connected to the laser light source 7, which is disposed outside the insertion portion 2, and laser light output from the laser light source 7 is input to the proximal end of the optical fiber 3a as the illumination light L.

The scanner 3b vibrates a distal-end section of the optical fiber 3a in directions intersecting the longitudinal direction of the optical fiber 3a, thereby scanning the illumination light L, emitted from a distal end of the optical fiber 3a, along a predetermined scanning trajectory. The scanning trajectory is, for example, a spiral form, a raster form, or a Lissajous form. The scanner 3b is, for example, a piezoelectric actuator that vibrates the distal-end section of the optical fiber 3a through expansion and contraction of piezoelectric elements or an electromagnetic actuator that vibrates the distal-end section of the optical fiber 3a by a magnetic force.

As the light-guide optical system 3, it is also possible to adopt a method for scanning the illumination light L by using a galvanometer mirror.

The illumination optical system 4 includes two spherical lenses 4a and 4b that are perfect spheres. The two spherical lenses 4a and 4b are arranged in a direction parallel to the longitudinal axis of the insertion portion 2, and the optical axis A of the spherical lenses 4a and 4b is parallel to the longitudinal axis of the insertion portion 2. The diameter of the spherical lens 4a, which is closer to the distal end, is smaller than the diameter of the spherical lens 4b, which is closer to the proximal end. The illumination light L emitted from the distal end of the optical fiber 3a passes through the two spherical lenses 4a and 4b and is radiated onto the subject S. The two spherical lenses 4a and 4b have a function to further widen the angle of the illumination light L to be scanned.

The optical waveguide 5 has a cylinder shape extending from the distal-end section 2a to the proximal-end section 2b, and a distal-end surface of the optical waveguide 5 is disposed at the distal end of the insertion portion 2. The optical waveguide 5 receives the observation light L′ at the distal-end surface and guides the observation light L′ toward the proximal-end section 2b. Specifically, the optical waveguide 5 functions as a light-receiving optical system that receives the observation light L′. The outer cover 8 is disposed on the outer circumferential surface (outer surface) of the optical waveguide 5 along the shape of the outer circumferential surface of the optical waveguide 5 and covers the outer circumferential surface of the optical waveguide 5. Accordingly, the optical waveguide 5 is protected by the outer cover 8 and is stably supported by the outer cover 8.

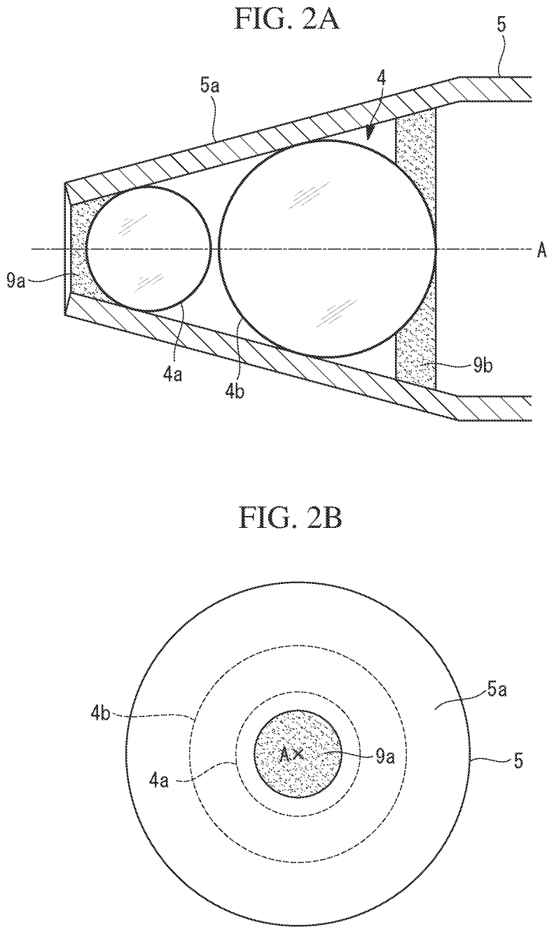

As shown in FIGS. 2A and 2B, a distal-end section of the optical waveguide 5, the distal-end section being disposed in the distal-end section 2a, is a tapered section 5a having a tapered shape so as to be gradually reduced in diameter, toward the distal end, and the two spherical lenses 4a and 4b are held inside the tapered section 5a. The diameter of an opening in the distal-end surface of the tapered section 5a is smaller than the diameter of the spherical lens 4a, and the spherical lenses 4a and 4b abut against an inner circumferential surface of the tapered section 5a over the entire circumferences thereof. With the tapered section 5a, the spherical lenses 4a and 4b are stably held inside the distal-end section 2a. Furthermore, because the tapered section 5a is disposed around the spherical lenses 4a and 4b over the entire circumferences thereof in the circumferential direction about the optical axis A, the observation light L′ can be received without being spatially biased.

A distal-end section of the lens surface of the spherical lens 4a is covered by an adhesive agent 9a, and the spherical lens 4a and the optical waveguide 5 are fixed to each other by the adhesive agent 9a. A proximal-end section of the lens surface of the spherical lens 4b is covered by an adhesive agent 9b, and the spherical lens 4b and the optical waveguide 5 are fixed to each other by the adhesive agent 9b. It is preferred that a distal-end surface of the adhesive agent 9a and a proximal-end surface of the adhesive agent 9b each be flat.

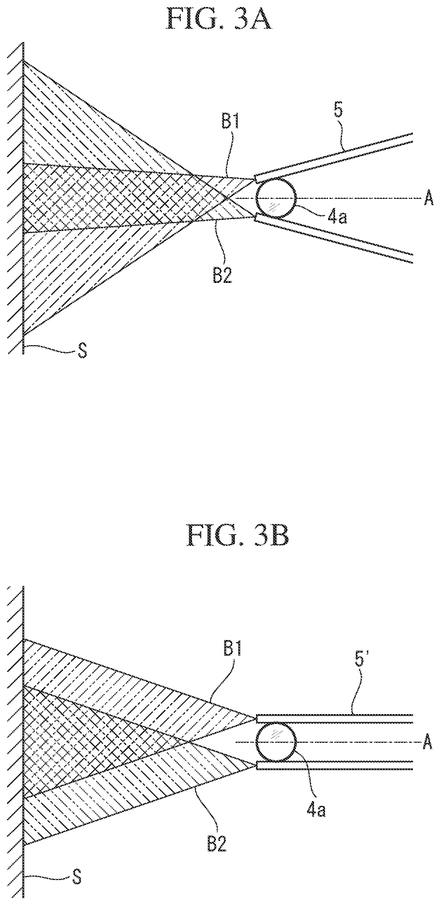

FIG. 3A explains light-receiving ranges B1 and B2 within which the optical waveguide 5 can receive the observation light L′. FIG. 3B explains light-receiving ranges B1 and B2 of an optical waveguide 5′ according to a comparative example. A distal-end section of the optical waveguide 5′ is parallel to the optical axis A.

The tapered section 5a is inclined in such a direction as to gradually approach the optical axis A of the spherical lenses 4a and 4b toward the distal end. Furthermore, in a normal endoscope design, the observation distance from the distal end of the insertion portion 2 (the distal end of the optical waveguide 5) to the subject S is substantially larger than the diameter of the insertion portion 2. Therefore, as shown in FIG. 3A, when the light-receiving ranges B1 and B2 of the optical waveguide 5, which are located at two positions opposed to each other in a radial direction, are considered, the two light-receiving ranges B1 and B2 intersect each other in the vicinity of the distal end of the insertion portion 2 and get away from each other in a radial direction orthogonal to the optical axis A, toward the subject S. On the other hand, as shown in FIG. 3B, the two light-receiving ranges B1 and B2 of the optical waveguide 5′ are parallel to each other.

In this way, with the tapered section 5a being provided, the light-receiving ranges on the subject S are expanded in the radial direction, and the optical waveguide 5 is made to have a wider angle, compared with the optical waveguide 5′.

The light detecting part 6 has a light receiving element such as a photodiode. The light detecting part 6 detects the intensity of the observation light L′ entering the light receiving element from the proximal end of the optical waveguide 5.

Information on the intensity of the observation light L′ detected by the light detecting part 6 is sent to an image processing device (not shown). The image processing device associates the positions of the illumination light L on the scanning trajectory with the intensities of the observation light L′ to form a 2D image of the subject S and displays the image on a display unit (not shown).

Next, the operation of the thus-configured endoscope device 1 will be described below.

According to the endoscope device 1 of this embodiment, the illumination light L output from the laser light source 7 is guided inside the insertion portion 2 from the proximal-end section 2b toward the distal-end section 2a by the light-guide optical system 3 and is radiated onto the subject S after the angle thereof is widened by the spherical lenses 4a and 4b in the distal-end section 2a. The illumination light L is scanned on the subject S by the scanner 3b, and the observation light L′ is generated at the positions, on the scanning trajectory, where the illumination light L is radiated. The observation light L′ is, for example, reflected light of the illumination light L or fluorescence excited by the illumination light L. Part of the observation light L′ generated at the subject S is received by the optical waveguide 5, is guided to the light detecting part 6, and is detected by the light detecting part 6. The observation light L′ at respective positions of the scanning trajectory on the subject S is detected by the light detecting part 6, and an image of the subject S is formed on the basis of the intensities of the detected observation light L′.

In this case, in order to expand the observation field of view of the endoscope device 1, both the illumination optical system 4 and the optical waveguide 5, which functions as a light-receiving optical system, need to have wide angles. According to this embodiment, the illumination optical system 4 is made to have a wide angle by the spherical lenses 4a and 4b, and the optical waveguide 5 is made to have a wide angle by the tapered section 5a. Specifically, it is possible to radiate the illumination light L onto a wide observation field of view on the subject S and to receive the observation light L′ from the wide observation field of view on the subject S. As a result, there is an advantage in that a wide observation field of view can be observed.

Furthermore, in an assembly process for the insertion portion 2, the outer surfaces of the spherical lenses 4a and 4b are made to abut against the inner circumferential surface of the tapered section 5a, thereby positioning the spherical lenses 4a and 4b so as to align the optical axis A of the spherical lenses 4a and 4b with the central axis of the optical waveguide 5. In this way, there is an advantage in that assembly of the optical waveguide 5 and the spherical lenses 4a and 4b can be easily performed.

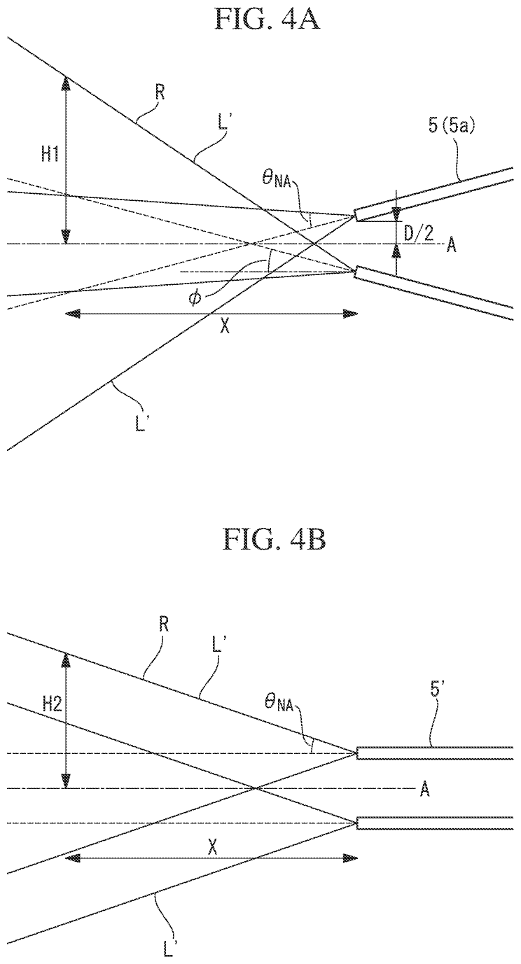

FIG. 4A explains the relationship between an inclination angle φ of the tapered section 5a with respect to the optical axis A and a light-receiving range H1 of the optical waveguide 5. FIG. 4B explains a light-receiving range H2 of the optical waveguide 5′.

It is preferred that the inclination angle φ (>0) of the tapered section 5a satisfy the following Expression (1). D indicates the diameter of the optical waveguide 5 at the distal end thereof. Specifically, D/2 indicates the distance between the distal end of the optical waveguide 5 and the optical axis A. X indicates the observation distance from the distal end of the optical waveguide 5 to the subject S. θ_{NA} indicates a one-side light-receiving angle of the optical waveguide 5. H1 and H2 each indicate a radius of the light-receiving range of the observation light L′ on the subject S. The inclination angle φ is designed so as to satisfy the following Expression (1). By satisfying Expression (1), the light-receiving range H1 of the optical waveguide 5 can be expanded, compared with the light-receiving range H2 of the optical waveguide 5′.

$$ϕ > {tan}^{- 1} ( \frac{D}{X {tan}^{2} {θ}_{NA} + D tan {θ}_{NA} + X} ) \qquad ( 1 )$$

It is further preferred that the inclination angle φ satisfy the following Expression (2). Xmax indicates the maximum value of an observation depth range. By satisfying Expression (2), it is possible to obtain an effect of expanding the light-receiving range H1 at least in a section of the observation depth range of the optical systems 4 and 5. Note that the observation depth range is a range between a near point and a far point in the depth of field of the endoscope device 1, and Xmax corresponds to the observation distance to the far point.

$$ϕ > {tan}^{- 1} ( \frac{D}{{X}_{max} {tan}^{2} {θ}_{NA} + D tan {θ}_{NA} + {X}_{max}} ) \qquad ( 2 )$$

Note that Expression (1) is derived as follows.

In FIGS. 4A and 4B, the light-receiving ranges H1 and H2 are determined by light rays R. The light ray R is a light ray located outermost in a radial direction orthogonal to the optical axis A, among light rays entering the optical waveguide 5 from the subject S. From the geometric relationships shown in FIGS. 4A and 4B, H1 and H2 are expressed as follows.

H1=X×tan(φ+θ_{NA})−D/2 (a)

H2=X×tanθ_{NA}+D/2 (b)

The condition for obtaining a wide angle effect due to the tapered section 5a is described in the following Expression (c):

H1>H2 (c)

Expression (1) is derived from Expressions (a), (b), and (c).

In this embodiment, although the spherical lenses 4a and 4b are used as the illumination optical system, instead of this, they may be used as the light-receiving optical system. In this case, the optical waveguide 5 is used as the illumination optical system.

Specifically, the illumination light L from the laser light source 7 is guided from the proximal end of the optical waveguide 5 toward the distal end thereof and is radiated onto the subject S from the distal end of the optical waveguide 5. The observation light L′ is received by the spherical lens 4a at the distal end of the insertion portion 2 and is guided toward the proximal-end section of the insertion portion 2 by the light-guide optical system. The light-guide optical system in this case is formed of, for example, a combination of a plurality of lenses. The light detecting part 6 is, for example, an image acquisition device and detects the observation light L′ guided by the light-guide optical system.

According to this configuration, the illumination optical system is made to have a wide angle by the tapered section 5a, and the light-receiving optical system is made to have a wide angle by the spherical lenses 4a and 4b. Therefore, a wide observation field of view can be observed.

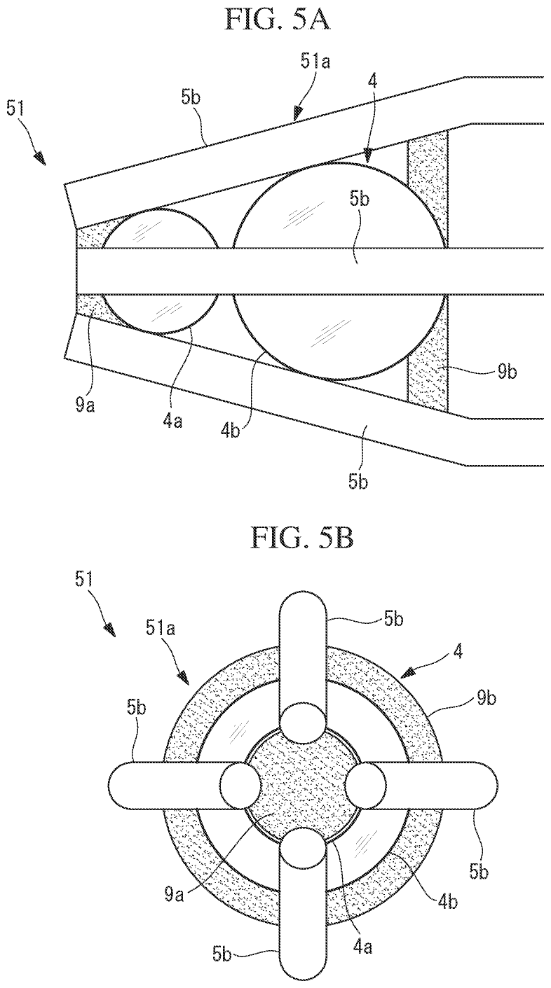

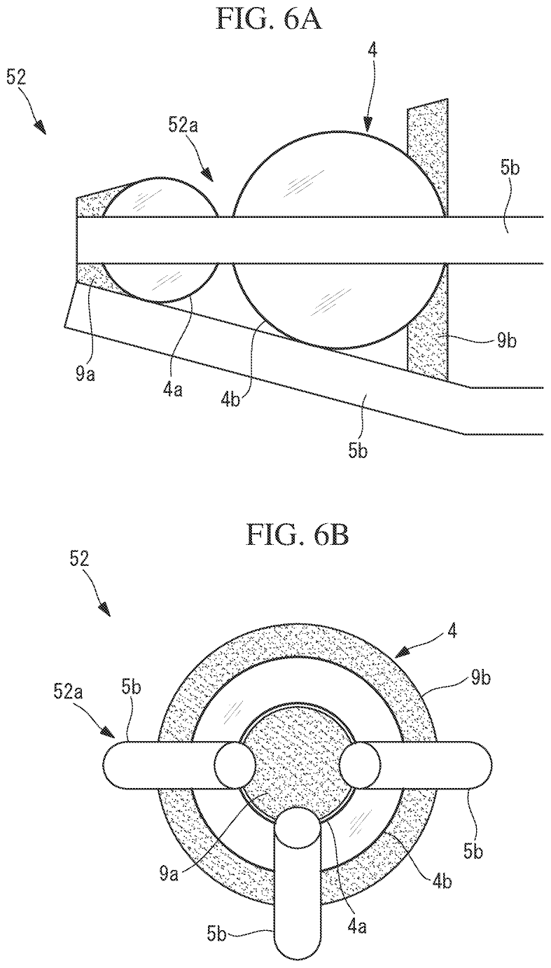

In this embodiment, although the cylindrical optical waveguide 5 is used, the specific configuration of the optical waveguide 5 is not limited thereto. FIG. 5A to FIG. 6B show modifications of the optical waveguide 5.

An optical waveguide 51 shown in FIGS. 5A and 5B is formed of a plurality of optical fibers 5b that are evenly arranged around the spherical lenses 4a and 4b over the entire circumferences thereof. The optical waveguide 51 shown in FIGS. 5A and 5B is formed of four optical fibers 5b. The number of optical fibers 5b may also be three or less or five or more. The optical waveguide 51 may also be formed of a plurality of fiber-shaped optical waveguides, instead of a plurality of optical fibers 5b. According to the optical waveguide 51, as in the optical waveguide 5, the observation light L′ can be received without being spatially biased.

Distal-end sections of the respective optical fibers 5b are inclined in such directions as to approach the optical axis A toward the distal end. A tapered section 51a is formed of the distal-end sections of the plurality of optical fibers 5b.

An optical waveguide 52 shown in FIGS. 6A and 6B is formed of a plurality of optical fibers 5b or a plurality of fiber-shaped optical waveguides, as in the optical waveguide 51. However, the plurality of optical fibers 5b are unevenly arranged around the spherical lenses 4a and 4b. A tapered section 52a is formed of the distal-end sections of the plurality of optical fibers 5b, as in the tapered section 51a.

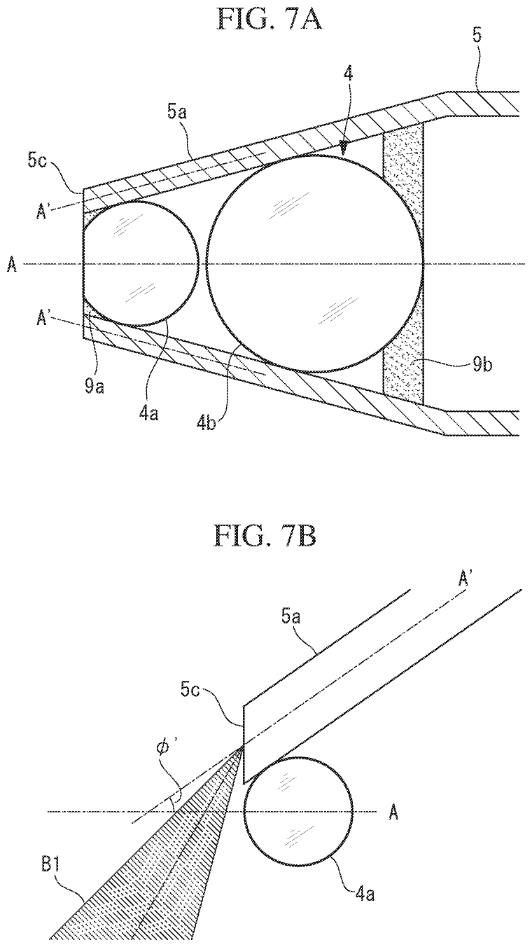

In this embodiment, as shown in FIG. 7A, a distal-end surface 5c of the optical waveguide 5 may also be inclined with respect to the optical axis A′ of the optical waveguide 5.

In the example case shown in FIG. 7A, the distal-end surface 5c is a flat surface perpendicular to the optical axis A. The distal-end surface 5c is formed by grinding, from the distal end, the assembly of the spherical lenses 4a and 4b and the optical waveguide, which have been fixed to each other. Therefore, a distal-end surface of the spherical lens 4a may also be a flat surface perpendicular to the optical axis A.

FIG. 7B explains the relationship between the inclination of the distal-end surface 5c with respect to the optical axis A′ and the inclination of a light-receiving range B1 with respect to the optical axis A. With the distal-end surface 5c being inclined with respect to the optical axis A′, the light-receiving range B1 is inclined more toward the optical axis A, compared with a case in which the distal-end surface 5c is perpendicular to the optical axis A′. Therefore, the optical waveguide 5 can be made to have an even wider angle. Furthermore, in a case in which the distal-end surface of the spherical lens 4a is a flat surface perpendicular to the optical axis A, the illumination light L can be efficiently emitted.

In the example cases shown in FIGS. 7A and 7B, the tapered section 5a is inclined at an inclination angle φ′(>0) with respect to the optical axis A, and the distal-end surface 5c is perpendicular to the optical axis A. At this time, it is preferred that the inclination angle φ′ satisfy the following Expression (3). In Expression (3), n indicates the refractive index on the axis of the optical waveguide 5. By satisfying Expression (3), the light-receiving range of the optical waveguide 5 can be expanded, compared with the light-receiving range of the optical waveguide 5′, in which the distal-end section thereof is parallel to the optical axis A.

$${ϕ}^{′} > {sin}^{- 1} \frac{1}{n} [ {tan}^{- 1} ( \frac{D}{X {tan}^{2} {θ}_{NA} + D tan {θ}_{NA} + X} ) ] \qquad ( 3 )$$

It is further preferred that the inclination angle φ′ satisfy the following Expression (4). By satisfying Expression (4), it is possible to obtain an effect of expanding the light-receiving range at least in a section of the observation depth range of the optical systems 4 and 5.

$${ϕ}^{′} > {sin}^{- 1} \frac{1}{n} [ {tan}^{- 1} ( \frac{D}{{X }_{max} {tan}^{2} {θ}_{NA} + D tan {θ}_{NA} + {X}_{max}} ) ] \qquad ( 4 )$$

Note that Expression (3) is derived as follows.

In FIG. 7B, the following Expression is established from Snell's law.

n×sin φ′=1×sin A (d)

Expression (d) can be rewritten to Expression (d′).

A=sin⁻¹(n×sin φ′) (d′)

In Expression (1), φ is replaced with A, thereby obtaining Expression (1′).

$$A > {tan}^{- 1} ( \frac{D}{X {tan}^{2} {θ}_{NA} + D tan {θ}_{NA} + X} ) \qquad ( {1}^{′} )$$

Expression (3) is derived from Expression (1′) and Expression (d′).

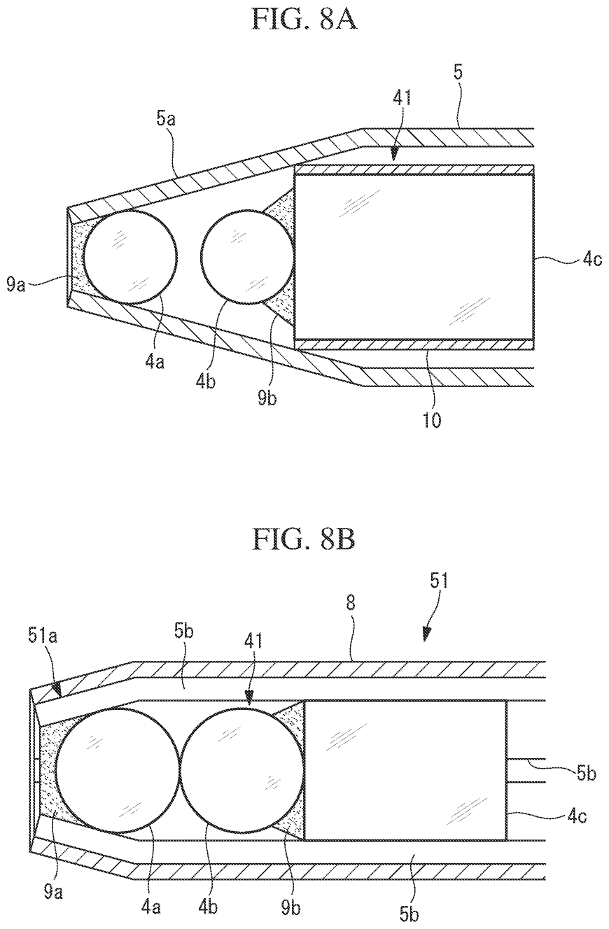

In this embodiment, as shown in FIGS. 8A to 8C, an illumination optical system 41 may further include an image transmission system 4c at the proximal end of the spherical lenses 4a and 4b. The image transmission system 4c is a gradient index (GRIN) lens, and the spherical lens 4b is fixed to a distal-end surface of the GRIN lens by the adhesive agent 9b. The GRIN lens 4c may also be a part of the light-guide optical system 3. The illumination optical system 41, which includes the image transmission system 4c, is suitable when the endoscope device is a rigid scope. The image transmission system 4c may also be formed of a combination of a plurality of lenses.

In a modification shown in FIG. 8A, the diameter of the image transmission system 4c is larger than the diameter of the spherical lens 4b. At the time of assembly, a distal end of a cylindrical outer frame 10 that holds the image transmission system 4c therein is made to abut against the inner circumferential surface of the tapered section 5a, thereby positioning the spherical lens 4b and the image transmission system 4c, which are integrated by the adhesive agent 9b, with respect to the optical waveguide 5. In a case in which the outer frame 10 is not provided, the distal end of the image transmission system 4c may also be made to abut against the inner circumferential surface of the tapered section 5a. With this configuration, because positioning can be performed in a state in which the inclination of the tapered section 5a is increased so as to suit to the diameter of the image transmission system 4c, the light-receiving optical system can be made to have an even wider angle.

In a modification shown in FIG. 8B, the two spherical lenses 4a and 4b are in contact with each other. The diameters of the spherical lenses 4a and 4b may also be equal to each other.

As shown in FIG. 8C, the insertion portion 2 may further include a cylindrical inner cover 11. The inner cover 11 is disposed between the optical waveguide 51 and the illumination optical system 41 and covers an inner surface of the optical waveguide 51, the inner surface being close to the illumination optical system 41. The inner cover 11 is, for example, a pipe made of metal, such as stainless steel, and has light-shielding properties and rigidity. The illumination optical system 41 and the optical waveguide 51 are spatially separated from each other by the inner cover 11. Therefore, it is possible to prevent a situation in which the illumination light L leaks from the illumination optical system 41 to the optical waveguide 51 and becomes mixed with the observation light L′. Furthermore, the illumination optical system 41 and the optical waveguide 51 can be stably supported by the inner cover 11, which is a rigid body. The inner cover 11 may also be a light-shielding sheet-like member that does not have rigidity or has low rigidity.

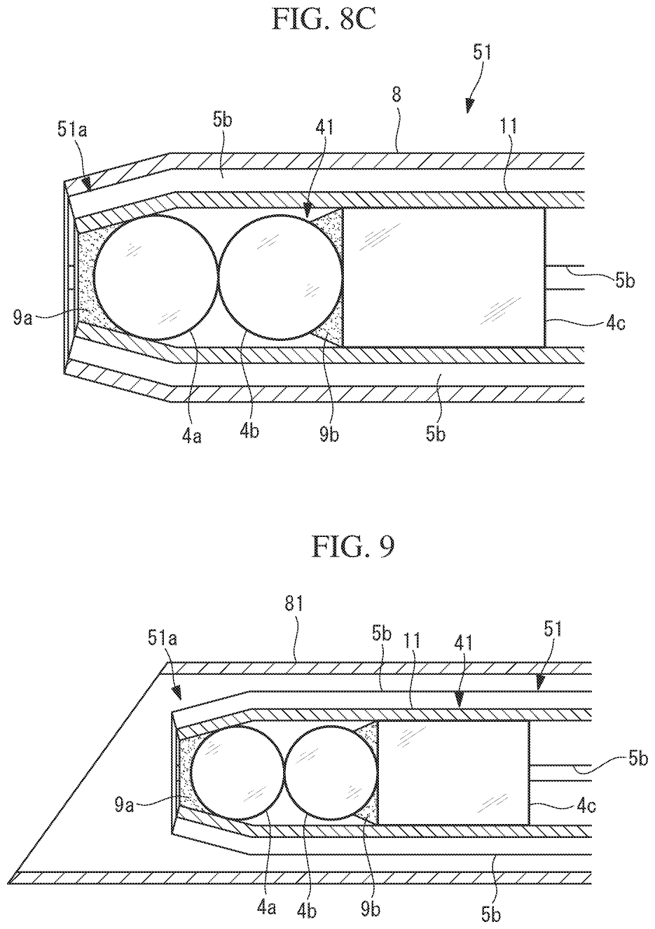

In this embodiment, instead of the outer cover 8, which is in close contact with the outer surface of the optical waveguide 5, 51, or 52, as shown in FIG. 9, it is also possible to adopt an outer cover 81 that has a larger inner diameter than an outer diameter of the optical waveguide 5, 51, or 52.

The outer cover 81 is made of metal and has rigidity. The outer cover 81 may also be a hollow needle having a distal-end surface inclined with respect to the longitudinal axis. The illumination optical system 41 and the optical waveguide 51 are movable inside the outer cover 81 in the longitudinal direction. A gap between the inner circumferential surface of the outer cover 81 and the outer surface of the optical waveguide 51 may also be used as a fluid passage.

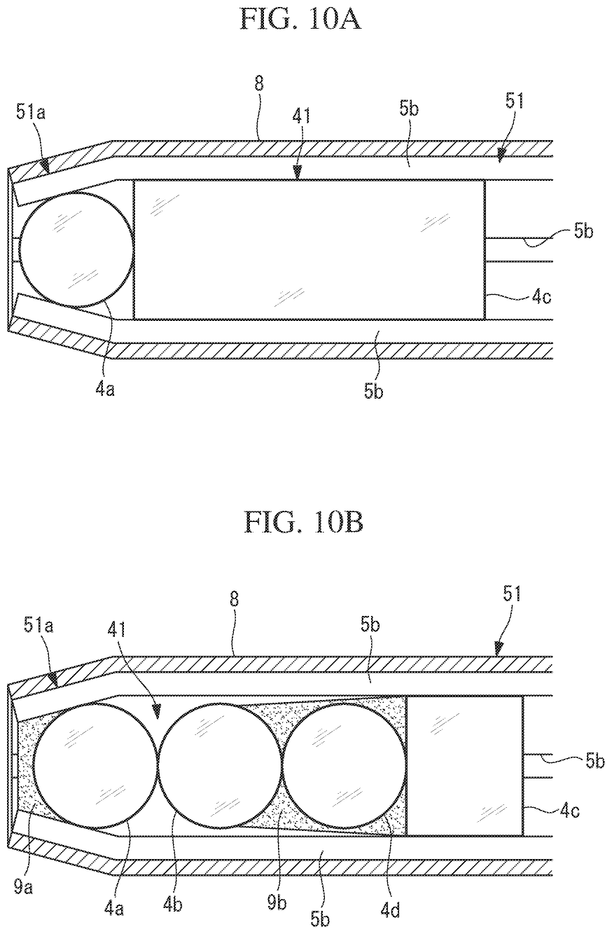

In this embodiment, although the illumination optical system 4 includes the two spherical lenses 4a and 4b, the number of spherical lenses may be only one, as shown in FIG. 10A. Alternatively, as shown in FIG. 10B, three spherical lenses 4a, 4b, and 4d or more may also be provided.

An adhesive agent on the lens surface of a spherical lens leads to reduction of refractive power. Therefore, in a case in which only one spherical lens is used, in order to ensure large positive refractive power of the entire illumination optical system, as shown in FIG. 10A, it is preferred that no adhesive agent be provided on a distal-end section and a proximal-end section of the lens surface of the spherical lens 4a.

In the above-described embodiment and modifications, although the endoscope device 1 is of a scanning type, instead of this, the endoscope device 1 may also be of a non-scanning type. For example, instead of the light-guide optical system 3, which has the optical fiber 3a and the scanner 3b, it is also possible to provide a light-guide optical system that is formed of a combination of a plurality of lenses or an optical-fiber bundle.

### REFERENCE SIGNS LIST

1 endoscope device
2 insertion portion
2a distal-end section
2b proximal-end section
8, 81 outer cover
3 light-guide optical system
3a optical fiber
3b scanner
4 illumination optical system
4a, 4b spherical lens
4c image transmission system, gradient-index lens (light-guide optical system)
5, 51, 52 optical waveguide
5a, 51a, 52a tapered section
5b optical fiber
5c distal-end surface
6 light detecting part
7 laser light source
9a, 9b adhesive agent
10 outer frame
11 inner cover
L illumination light
L′ observation light
A optical axis of spherical lens
A′ optical axis of optical waveguide

1. A scanning endoscope comprising:
a long insertion portion that has a distal-end section and a proximal-end section;
a light-guide optical system that guides illumination light coming from a light source toward the distal-end section;
a spherical lens that is disposed in the distal-end section and that radiates the illumination light guided by the light-guide optical system onto a subject;
an optical waveguide that extends from the distal-end section to the proximal-end section, that receives observation light coming from the subject, and that guides the observation light; and
a light detector that detects the observation light guided by the optical waveguide,
wherein the optical waveguide is inclined, at the distal-end section, in such a direction as to approach an optical axis of the spherical lens toward a distal end.

2. The scanning endoscope according to claim 1, wherein the optical waveguide is disposed at an outer side of the spherical lens in a radial direction orthogonal to the optical axis of the spherical lens and is disposed entirely in a circumferential direction about the optical axis of the spherical lens.

3. The scanning endoscope according to claim 1,
wherein the light-guide optical system comprises a gradient index lens, and the spherical lens is fixed to a distal-end surface of the gradient index lens; and
wherein a distal end of the gradient index lens or a distal end of an outer frame that holds the gradient index lens abuts against a surface of the optical waveguide, the surface being close to the spherical lens.

4. The scanning endoscope according to claim 1,
wherein the insertion portion comprises a cylindrical rigid outer cover that forms the outermost circumferential surface of the insertion portion; and
wherein the outer cover covers an outer surface of the optical waveguide, the outer surface being located at an opposite side from the spherical lens.

5. The scanning endoscope according to claim 4, wherein the outer cover is disposed on the outer surface of the optical waveguide along the shape of the outer surface.

6. The scanning endoscope according to claim 4, wherein the insertion portion comprises a light-shielding inner cover that covers an inner surface of the optical waveguide, the inner surface being close to the spherical lens.

7. The scanning endoscope according to claim 6, wherein the inner cover is a rigid body.

8. The scanning endoscope according to claim 1, wherein a distal-end surface of the optical waveguide is inclined with respect to an optical axis of the optical waveguide.

9. A scanning endoscope comprising:
a long insertion portion that has a distal-end section and a proximal-end section;
an optical waveguide that extends from the distal-end section to the proximal-end section, that guides illumination light coming from a light source toward the distal-end section, and that radiates the illumination light onto a subject;
a spherical lens that is disposed in the distal-end section and that receives observation light coming from the subject;
a light-guide optical system that guides the observation light received by the spherical lens; and
a light detector that detects the observation light guided by the light-guide optical system,
wherein the optical waveguide is inclined, at the distal-end section, in such a direction as to approach an optical axis of the spherical lens toward a distal end.
